(19) United States
(12) Patent Application Publication
(10) Pub. No.: US 2024/0000596 A1
(43) Pub. Date: Jan. 4, 2024

(54) Ankle Orthosis

(71) Applicant: Icarus Medical, LLC, Charlottesville, VA (US)

(72) Inventors: David T. Johnson, Charlottesville, VA (US); Evan Eckersley, Charlottesville, VA (US); Ben Scire, Hopkinton, MA (US); Carter Kitchin, Charlottesville, VA (US); Philip Miller, Charlottesville, VA (US)

(73) Assignee: Icarus Medical, LLC, Charlottesville, VA (US)

(21) Appl. No.: 18/144,796

(22) Filed: May 8, 2023

Related U.S. Application Data
(63) Continuation-in-part of application No. 17/537,476, filed on Nov. 29, 2021, now Pat. No. 11,666,472.
Application No. 17/537,476 is a continuation-in-part of application No. 17/211,635, filed on Mar. 24, 2021, now Pat. No. 11,819,436.
Application No. 17/211,635 is a continuation of application No. 17/074,542, filed on Oct. 19, 2020, now Pat. No. 11,564,824.
Application No. 17/074,542 is a division of application No. 15/585,968, filed on May 3, 2017, now Pat. No. 10,806,619.
Application No. 17/537,476 is a continuation-in-part of application No. 17/074,571, filed on Oct. 19, 2020.
Application No. 17/074,571 is a continuation-in-part of application No. 15/585,968, filed on May 3, 2017, now Pat. No. 10,806,619.
(60) Provisional application No. 62/331,315, filed on May 3, 2016.
(60) Provisional application No. 62/331,315, filed on May 3, 2016.

(51) Int. Cl. A61F 5/01

(52) U.S. Cl. CPC A61F 5/0125; A61F 2005/0165; A61F 2005/0139

(57) ABSTRACT

A multi-axis rotation control ankle brace, wherein the direction and magnitude of force can be controlled around three axes of the ankle joint through an adjustable tensioning apparatus. The device may be applied to address conditions such as chronic ankle instability, foot drop, or osteoarthritis by providing such forces around the joint as an external-muscle tendon system to improve function, reduce pain, or restore mobility of the user. While more are contemplated herein, five preferred embodiments are specifically disclosed in the current application. Three preferred embodiments comprise a proximal portion and a distal portion, wherein the proximal portion is anchored above the ankle joint and houses, in aspects, the adjustment mechanism. The proximal portion is connected to the distal portion by tensioning or compressive elements, through which threes can be controlled by the user via the adjustment mechanism. In other preferred embodiments, the device is comprised of one continuous mesh, sock or sleeve through which tension can be controlled by the user. In other preferred embodiments, the device is personalized to the user through multiple aspects including user-enabled adjustment of forces around the joint. The device may be customized by 3D printing a device based on a digital scan, and therefore conforms to the user's ankle and foot.

### CROSS-REFERENCE TO RELATED APPLICATIONS

The present application is a continuation-in-part of and claims priority to U.S. application Ser. No. 17/537,476 filed Nov. 29, 2021, which is a continuation-in-part of and claims priority to U.S. application Ser. No. 17/211,635, which is a continuation of and claims priority to U.S. application Ser. No. 17/074,542 filed Oct. 19, 2020, which is a division of U.S. application Ser. No. 15/585,968 filed May 3, 2017, now U.S. Pat. No. 10,806,619 issued Oct. 20, 2020, which claims benefit of Provisional Patent No. 62/331,315 filed May 3, 2016.

The present application is a continuation-in-part of and claims priority to U.S. application Ser. No. 17/537,476 filed Nov. 29, 2021, which is a continuation-in-part of and claims priority to U.S. application Ser. No. 17/074,571 filed Oct. 19, 2020, which is a continuation-in-part of and claims priority to U.S. application Ser. No. 15/585,968 filed May 3, 2017, now U.S. Pat. No. 10,806,619 issued Oct. 20, 2020, which claims benefit of Provisional Patent No. 62/331,315 filed May 3, 2016.

The present application is a continuation-in-part of and claims priority to U.S. application Ser. No. 17/537,476 filed Nov. 29, 2021, which relates to and claims priority to and the benefit of the filing date of PCT/US2020/047904, filed August 26, 2020.

The disclosures of all applications referenced above are hereby incorporated by reference herein in their entireties.

### BACKGROUND OF THE INVENTION

### Field of the Invention

The present invention disclosed herein relates to an ankle orthosis comprising an adjustable tensioning apparatus designed to control rotation of the ankle joint around one or more axes.

### Description of Related Art

The ankle presents one of the more complicated joints within the body, as well as one facing a diverse range of prevalent indications. Individuals may be affected by conditions ranging from ankle sprain to foot drop to osteoarthritis. Each condition may arise due to a range of underlying causes including a chronic condition from prior injury, a neurological disorder, or general musculoskeletal deficiencies. For example, 3.6% of the population experiences ankle sprains annually within the US. Most individuals experiencing ankle sprains pursue self-treatment options such as the PRICE method (protection, rest, ice, compression, and elevation). Immobilization in the form of bracing is often pursued. Due to self-treatment, sprains are largely underreported because most patients never visit a clinic or professional.

While this injury may seem temporary to the individual at the time of the sprain, it presents serious long-term implications. Such indications include chronic ankle instability (CAI) resulting in recurring sprains, tendon and ligament damage, osteoarthritis, and fracture. Most sprains (70%) involve a lateral sprain (inversion). While most ankle sprains are successfully managed with nonoperative modalities, recurrent instability and associated defects can be seen in 25-40% of patients, which only accounts for those reported in a largely unreported field. The incidence of residual symptoms following acute ankle sprain is variable, but has been reported with rates between 40-50%. Additionally, 40-70% of patients sustaining a lateral ankle sprain may develop CAI, characterized by residual symptoms of the ankle ‘giving way’ and feelings of ankle joint instability for at least one year following the initial sprain. The condition ultimately leads to insufficiency of the lateral ankle ligament complex. This negatively alters central mechanisms of motor control, leading to an increased risk of falls, and is a leading cause of post-traumatic ankle joint osteoarthritis. It also affects the user's gait and creates a compounding effect on biomechanics of other lower limb joints, including the knee, hip and back. Ultimately, a single sprain may result in a significantly reduced quality of life, which is associated with increased mortality due to comorbidities such as heart disease, stroke, depression and diabetes.

While the indications facing the ankle joint are varied, numerous, and prevalent, there is a lack of personalized solutions. For example, in the event of ankle fracture or osteoarthritis, a common solution is ankle arthrodesis (ankle fusion). In this invasive procedure, the bones of the joint are fused into one piece. The median cost of the procedure is over $40,000. While the procedure may reduce pain within the joint in the short term by eliminating the articulation of arthritic surfaces, the procedure presents lasting challenges including lack of mobility of the ankle, reduced stability, and reduced function. Additionally, the changes to the individual's gait due to reduced joint flexibility lead to adverse biomechanical forces in other joints of the lower body including the knee, hip, and back. As a result, the individual's overall mobility health and long-term quality of life declines.

While fusion surgery is used in extreme cases, ankle braces present an alternate solution to treating ankle instability and osteoarthritis. Many existing ankle braces focus on immobilizing the joint completely. While this avoids the cost associated with fusion surgery, these braces are less effective, yield mixed opinions between prescribers as to whether they are effective or not, present the same problems in limiting mobility in all axes of the joint, and do not present a personalized solution to the unique needs of the individual.

Ankle braces are designed for a multitude of functions and indications in daily life. They may prevent foot drop, assist plantarflexion, or stabilize the joint. It is typical for an ankle brace to prevent exaggerated motion in a direction of the ankle joint, such as medial/lateral rotation or plantar/dorsiflexion, in order to prevent injury during use of the brace or help the user recover from an injury. While limiting range of motion in certain aspects, the ankle brace should minimally interfere with the user's daily activity. Although current braces may be able to address specific indications and limit movement to prevent further injury and/or support recovery while activity is maintained, they are often bulky, rigid, and not customized to address the user's specific deficiency or need. Additionally, they are often static devices that are not adjustable by the user or clinician to best address the user's need, desired activity or state of movement. There exists a major market need for a dynamically adjustable ankle brace that can address a series of indications in a low-profile design, which does not interfere with other daily functions of the user. According to the current invention, the directionality and magnitude of corrective forces to support, augment, and limit joint movement can be tailored to optimally address the user's need and desired function.

In regards to specific currently available ankle braces, Ultra Ankle® currently sells three models of their ankle brace, two models for moderate support and one model for maximum support. These braces vary from being form-fitting to more rigid, and are recommended to users based on how many prior ankle injuries they have had. While these braces do have an adjustable strap, they do not offer a method for adjusting tension to control rotation around the ankle. Furthermore, this brace only allows movement along one axis allowing plantar- and dorsiflexion. The maximum support version of this brace offers a removable cuff for extra support during rehabilitation from an ankle injury, that may be removed when more mobility is desired.

DonJoy® performance offers a variety of ankle braces, comprising a variety of materials ranging from rigid cuffs to slip-on materials. While DonJoy sells ankle braces for a variety of needs, it is important to notice that most of their adjustable braces comprise a strapping or lacing system for tightening the orthosis on the joint, which is unlike the current invention using a tensioning apparatus that can quickly be adjusted via an adjustment mechanism across one or a multiple of axes, allowing for precise control of the force direction and magnitude by the user.

U.S. Pat. No. 9,707,118 describes a boot-like AFO designed for children with a tension element that has limited adjustability. Unlike the multi-axis rotation control brace disclosed herein, the device described in U.S. Pat. No. 9,707,118 provides a constant low force along one axis of rotation to pull the foot into a dorsiflex position while at rest for the purpose of improving range of motion and preventing heel cord shortening. In addition, the elastic band of U.S. Pat. No. 9,707,118 is not meant as a dynamic energy storage element to provide control of the ankle joint's rotation, but to provide a constant force to stretch the foot into a predetermined position when at rest.

Other currently available products in the field serve to protect or rehabilitate the ankle through a variety of methods. More specifically, some braces have introduced components under tension around the ankle joint. One on market technology, the Sutti Bounders by Fabtech Systems, incorporate an elastic band at the heel of the device. This device is used only for the pediatric market for strengthening, as the device is not designed to address conditions such as foot drop, ankle osteoarthritis, ankle instability or other common indications. The device lacks a dynamically adjustable tensioning apparatus, as is described in the current invention. The direction of force cannot by changed, and rotation can only be controlled around the ML axis statically in one direction. Additionally, the design is limited to a low-power elastic band, which is insufficient to support the range forces required to address a range of indications and patient populations beyond pediatric strengthening. The embodiments of the multi-axis rotation control brace described herein are designed to support high forces significant enough to treat these broader patient populations while providing for dynamic adjustability by the user.

US20180333285A1 describes an apparatus for a human orthosis for control of foot drop. The application teaches that by rotating a dial, “the upper retention structure is configured to tighten around an anatomy of the user when the sock moves into a plantar flexed position”, so the resistance of the strap tightening circumscribing the calf is used to limit plantar flexion. This is different than the device disclosed herein, where, in aspects, the tensioning system applies a force directly to the distal portion, or to an elastomeric portion attached to the distal portion, and the direction of that force is modifiable by placement of anchor points, enabling control on multiple axes. Unlike the multi-axis rotation control brace described herein, this application does not demonstrate adjustability of the direction of force, or application of the mechanism for conditions beyond foot drop. In other words, the device cannot be tailored by a clinician or the user based on the specific direction and magnitude of force required for the user. Additionally, the multi-axis rotation control brace described herein incorporates tensioning and compression systems, which contain energy storage elements, e.g. an elastomer or a spring. Such elements provide more natural control of the ankle joint's rotation, having similar properties to those of tendons, ligaments and muscles.

The same limitations apply to an on-market technology, the SaeboStep® brace. The device is further limited, as it is not custom (off-the-shelf or one size fits all) and is not personalized to the user's need, as described in the multi-axis rotation control brace described herein. The SaeboStep has no distal portion, as described herein.

A Tamarack joint is a type of hinge joint commonly used in ankle orthoses (AFOs) to allow controlled motion in a specific direction. The Tamarack joint is typically positioned on the lateral or medial side of the ankle brace or AFO and allows for controlled dorsiflexion and plantarflexion of the ankle joint. The Tamarack joint is made up of two plates or leaves that are connected by a pivot, which allows the joint to bend in one direction but not in the other. When the wearer's foot moves in a certain direction, the Tamarack joint allows the AFO to flex or extend, providing support and stability to the ankle joint. The joint is designed to be flexible and durable, and it can be adjusted or replaced as needed to ensure proper alignment and function of the AFO. It is commonly used in ankle orthoses to help individuals with ankle injuries or conditions, such as ankle sprains, chronic instability, or foot drop. Overall, it can be added as a component to provide support, mobility, and protection to the ankle joint.

While Tamarack flexure joints have many benefits in ankle foot orthoses (AFOs), they also have some limitations that should be taken into consideration when designing and using these devices. Some of the limitations of Tamarack flexure joints in AFOs include: Limited range of motion: Tamarack flexure joints are designed to provide a limited range of motion in a specific direction, typically in the sagittal plane. This means that they may not be ideal for individuals who require a greater range of motion or more complex movement patterns. Limited adjustability: While Tamarack flexure joints can be adjusted to some extent, they are not as easily adjustable and require a swapping of components using specific tools, often by a certified orthotist. The user cannot adjust the function of the device, including the support provided and range of motion allowed, while wearing the orthosis. This in turn may limit optimal performance and comfort. Tamarack flexure joints can be prone to fatigue and failure over time, partially due to their lack of design to facilitate a range of activities. They may require regular replacement or maintenance of the joint, which can be inconvenient and costly. Lastly, these joints are vulnerable when subjected to shear forces, are designed primarily for flexion and extension, and may not provide as much resistance to torsion and shear forces as other types of joints. This may make them less effective for individuals who require more stability and support in more than one plane of motion. Overall, while Tamarack flexure joints have many benefits in AFOs, they may not be ideal for all individuals or applications.

### SUMMARY OF THE INVENTION

The invention disclosed offers the benefits of a flexure joint while also addressing the limitations of the device, providing a user-adjustable mechanism to control forces around the joint to meet the user's specific need.

The device described herein is a multi-axis rotation control ankle orthosis that can control forces around the ankle joint, as well as be customized to the user's needs in various aspects. It can be adjusted by the user, manufacturer or clinician through multiple features including: 1) a tensioning or compression apparatus where the user can adjust the magnitude and direction of corrective force based their activity or indication, 2) optionally, a mechanism for changing the direction/orientation of corrective force or forces through a series of anchors, anchor points, channels, slots, or other positioning or connecting elements, and 3) optionally, a method to custom-fabricate the shape or form the device to the user's anatomy to optimize the comfort and efficacy.

In some embodiments, the ankle orthosis is designed to limit motion in a specific direction, for example to prevent over rotation and counter rotation of the ankle joint; this is accomplished by applying different amounts of tension or compression around different regions of the ankle, and between some part of the foot or footwear and a part of the ankle, the proximal part of the ankle, or a part of the leg situated above the ankle. Tension is applied to manipulate the position of the foot relative to the tibia and fibula. The applied tension can be fixed, adjustable, or dynamically adjustable in that tension increases with increasing motion between the ankle-foot-leg system. In other embodiments, the same directional forces may be applied to augment motion of the foot-ankle complex in a desired direction. Through this mechanism, the brace allows mobility within certain axes while limiting it in others to maximize the freedom of the joint while providing only the necessary directional stability.

Some embodiments comprise a proximal portion and a distal portion, where the proximal portion is affixed above the ankle joint and houses the adjustment mechanism, in aspects. The proximal portion is connected to the distal portion by tensioning or compressive elements, through which forces optionally can be controlled by the user via the adjustment mechanism. In other embodiments, the device is comprised of one continuous mesh, sock or sleeve through which tension can be controlled by the user. In other embodiments, tension is generated between the proximal and distal portions of the brace or sock without a tension-adjusting mechanism. The amount of tension may be tailored to an individual's needs. The tensioning or compressive elements may be external, or partially- or fully-integrated within the brace or sock.

The device applies a tension that can hold the ankle and foot in a position that prevents or reduces the likelihood of injury, such as an ankle roll, or can prevent or reduce the likelihood of overuse injuries such as repeated over-supination or over-pronation. The device can also address conditions like foot drop by applying a significant force in the appropriate direction to counteract the front of the foot from dropping down. The foot drop device, for example, may generate a force across more than one axis for users who have a combination of foot drop and eversion. The same mechanism, applied differently may also augment activity in those with muscle or neurological deficiency, e.g. augmenting plantarflexion or dorsiflexion.

The described device may be customized to optimally address the user's needs. This includes features such custom fabrication from a 3D-scan or measurements, custom fit through modification by a certified professional (e.g. thermoforming), adjusting the amount of force applied in a given direction using an adjustment mechanism connected to a tensioning element (e.g. containing elastic bands) or compression element (e.g. containing springs), or changing of the direction in which force is applied, including the axis about which a moment is generated. Tensioning or compression elements may be in the form of bands, sheets or lines and integrated within or external to the brace or sock. The size and geometry of these elements may be tailored to yield specific mechanical properties. In elastic and elastic components may be combined in patterns or arrangements to control tension in areas where it is desired.

Mechanism for Adjusting the Magnitude of Force

The tensioning apparatus on the device allows for multiple aspects of customization for adjustment by the user, manufacturer or a certified professional (e.g. a doctor or physical therapist) for desired function, whether limitation of movement, augmentation of movement for rehabilitation, or performance enhancement. Among others, a novel aspect of the device relates to the ability to dynamically adjust the amount of tension in one or more directions around. the joint described herein as a multi-axis rotation control device. The term multi-axis means that tension can be generated across at least one axis. The amount of force generated in a given direction may be controlled dynamically or statically via energy storage elements in compression or tension. Energy storage elements are described as any element or component that stores mechanical energy including, for example, elastomeric bands, webs or other geometries, springs, pneumatic elements, electromagnetic elements, pneumatics, or any component that may act as a mechanical energy storage element that may be adjusted to hold variable forces. In aspects, the force within the element will be set by the user at a certain position, and will change throughout a range of motion.

For example, a series of bands on the lateral side of the ankle may be placed under tension to prevent inversion of the ankle to address issues of chronic ankle instability. In the same way, a compressive element (e.g. a spring) placed on the medial side of the ankle may also limit or prevent inversion of the ankle to address the same indication. By adjusting the magnitude of force within the energy storage element, the user can compensate for ligament, tendon, muscular or neurological deficiencies, for example.

The user can adjust the magnitude of force within the tensioning or compression element using an adjustment mechanism. An adjustment mechanism allows the user or wearer of the device to increase, decrease or remove the amount of force stored within the tension or compression element and therefore the forces generated around the ankle joint. In aspects, the adjustment mechanism may comprise a dial, lever, ratchet and pawl system, pulley, electric motor, to modify and control the amount of force supplied. The amount of force may be recommended by a prescriber or other professional and indicated on the device. The range of forces able to he applied by the mechanism may be prescribed by a professional, e.g. forces of 5-10 pounds may be achieved in the element through a construction or inclusion of certain components by the manufacturer or professional to support a rehabilitation regiment in line with the individual's need. Other patients may benefit from less than 5 pounds or more than 10 pounds of tension. In other words, the device may incorporate tensioning elements of varying storage modulus, size, cross section, material or mechanical properties in general. By example, a tensioning element that may be adjusted with forces between 0-1 pound. to assist in dorsiflexion may be used for weeks one and two of a rehabilitation regiment. The element may be replaced as rehabilitation progresses with bands of higher storage modulus, e.g. one that can hold up to 5 pounds of tensile force to aid in muscle strengthening.

In aspects, the user may be able to rapidly and conveniently adjust the device during activity. For example, the user may engage tension in the device while walking, increase tension further while walking uphill, and disengage tension while seated within a span of seconds. The tensioning apparatus may also have preset values or ranges. The tensioning apparatus may be adjusted manually or automatically via a system of at least one sensor and motor or actuator.

The adjustment mechanism may provide a range of mechanical advantage depending on the design selected for the specific use case. For example, an adjustment mechanism providing a mechanical advantage of 2:1 may be used in conjunction with tensioning elements of a lower spring constant and for applications in which a lower amount of force is required to correct the ankle. In other examples, an adjustment mechanism with a 12:1 mechanical advantage may be selected in conjunction with higher durometer elastomers or for a high-torque application of the tensioning device, such as stabilizing the ankle during activity. A range of mechanical advantage from 2:1 to 24:1, by example, may be achieved in a dial-based adjustment mechanism. This mechanical advantage may be achieved in a compact design with a gear system which may include planetary gear systems, worm gears, or cycloidal gears and combinations thereof. Alternatively, pulley systems incorporated into the device may provide a mechanical advantage in the adjustment mechanism.

The adjustment mechanism may be used by the individual to optimize for a certain activity, to be used at a certain stage of rehabilitation, or based on the current medical condition (e.g. if the user has a greater degree of pain on that day, they may add more tension to the element to further unload the joint). The degree of force generated or stored within the tensioning or compressive element may be modified based on a user-controlled dial. This allows the user to change the force from zero to maximum based on activity. The degree of unloading may be modified by interchangeable elements, e.g. springs or bands of different spring constants. In addition to being able to adjust tensile force from zero to maximum continuously to achieve any force within the range, the device may allow for tensioning to specific discrete forces (e.g. in 0.1 lb., 0.2 lb., 0.3 lb., 0.4 lb., 0.5 lb., 0.6 lb., 0.7 lb., 0.8 lb., 0.9 lb., 1.0 lb., 1.1 lb. increments), or may simply toggle between on and off (e.g. minimum and maximum force). The force may be adjusted in any number of increments including on or off, stepwise between a minimum and maximum force, or continuously on a gradient.

The tensioning elements may be constructed of different materials to provide different properties based on the user need. For example, a tensioning element consisting of an elastomeric element of certain viscoelastic properties may better mimic the feel of natural muscle, tendon, and ligament complexes, while a more rigid or fully rigid tensioning element may completely lock the foot in one or more directions or prevent movement beyond a specific range of motion.

Mechanism for Adjusting the Direction of Force

The direction of force may be modified statically or dynamically by the user, manufacturer, or a certified individual to address a specific need. In one aspect, a tensioning element may be oriented on the lateral side of the ankle or foot to address chronic ankle instability, unload the medial part of the ankle, or prevent inversion. See FIG. 1 for labeling conventions of the 3 axes of the ankle joint. In this embodiment, a torque applied about the AP axis would support a weak ankle (e.g., weak ligaments within the ankle, inferior muscle strength, and/or bone or cartilage damage). The anchor may be connected at anchor points between the two elements: one at the proximal cuff and one or more at the distal portion, which yield a net force generated around the AP axis to prevent inversion. Multiple anchor points may be distributed along the proximal or distal cuff to provide greater stability and force distribution. This distribution may be created as a function of the geometry of the tensioning or compression element, which yields a net force vector controlled through one or more adjustment mechanism that optimally reduces inversion or eversion. For example, the shape and position of an elastomeric band, sheet or web between the proximal and distal portions will determine the force magnitude, direction, and overall distribution depending on the path it generates between the proximal portion and distal portion. The cables or tensioning elements may be routed through rigid or flexible material, or along the side or surface of the brace or sock to generate the desired force profile across the proximal and distal portions of the device. For example, the device meant to prevent foot drop will have cables and/or tensioning elements routed near the front of the foot above the ankle that have anchor points in the distal portion of the device, in a position that is forward from the position where the cables or tensioning elements connect in the proximal portion of the device.

In other aspects, the device may support dorsiflexion, for example to address foot drop, by being anchored to the front part of the foot or footwear and generate an adjustable torque along the ML axis. The user can increase tension to provide additional support for dorsiflexion and offset the muscle deficiencies that cause foot drop to restore a normal gait.

The tensioning or compression element may be anchored between the proximal and distal. portions to limit or support abduction or adduction. For example, if an individual had insufficient adduction, the element may he placed on the medial side of the foot, aligned with the tibialis anterior muscle, to supplement this movement.

As a multi-axis, rotation control device, the tensioning or compression elements may be anchored in a way that supports multiple directions of movement in the face of multi-directional deficiency. For example, it is common that individuals with foot drop also experience difficulty in adduction. In this instance, a tensioning element may be anchored on the lateral side of the proximal portion and the medial side of the distal portion. By generating force across this element through tensioning, the individual's joint position at rest will be restored to the normal position. The anchor point in either direction can be modified depending on the severity of muscle deficiency or need for correction in either direction. FIG. 3A shows one embodiment of a multi-axis rotation control orthosis to achieve this function. In this case, the device supplements movement around all three axes (the vertical, ML and AP axes), which is often the case in individuals with ankle conditions, due to the natural orientation of muscles around the joint. In this case, the device augments the tibialis anterior, which is often weakened in individuals with foot drop. However, there are multiple other muscles, ligaments, and tendons with different orientations and anchoring along the foot-ankle complex that may need to be augmented. A dynamically adjustable device allows each user's specific need to be addressed optimally in a single device.

In the proximal cuff, the tensioning elements may all be anchored at one point directly or indirectly to the adjustment mechanism. However, the direction of force and relative tension in each of the one or more tensioning elements may be modified by channels, grooves, guides or other structures on the proximal cuff, herein described generally as channels (see, e.g., FIG. 3A and FIG. 3B).

The anchor points may be determined to optimize function through algorithms, artificial intelligence, machine learning, and general software-automated design of the fabricated device.

The anchor points or connection of the tensioning element may be modified and placed at any point or angle of the proximal or distal portion of the device. There may be unlimited orientations and positions across the surface of the device. Alternatively, the anchor points may be pre-set to limit options for force direction. For example, the tensioning element may be anchored to position A to prevent inversion, or it may be placed at position B to do all of the following: limit inversion, support abduction and support dorsiflexion (see FIG. 3A and FIG. 3B for example).

Adjustment Mechanism

In order to control forces and rotation around one or more axes, the device may contain multiple tensioning or compression elements to address the user's need. In aspects, one or more tensioning or compression elements may be controlled by a single adjustment mechanism. The tensioning elements can be modular and added as needed based on the user's condition. Additionally, the device can contain one or more adjustment mechanisms of the same or varying design (e.g. rotary dials, levers, ratchet and pawl mechanisms, or motors) that change the magnitude of force within the tensioning or compression element(s) around one or more axes. The adjustment mechanisms may also change the direction of force by moving anchor points, changing tensioning or compression element(s) geometry, or changing orientation of the tensioning or compression element(s).

The user may be able to remove the adjustment mechanism, or may be able to move the adjustment mechanism from one point on the device to another. Multiple adjustment mechanisms may be incorporated on the device to modify force in multiple directions. The adjustment mechanism may be in the form of a slot with a corresponding external component, e.g. a key that can be inserted at multiple locations of the device to adjust forces in different orientations, engage or disengage different tensioning or compression elements, or change the direction of force.

Custom Fit

The device may be further customized through a user-specific fit. The shape of any of the proximal portion, distal portion, sock, or tensioning or compression element may conform to the user's body. A 3D scan may be used to manually, automatically, or semi-automatically fabricate a custom device, for example through 3D printing, or additive manufacturing, or subtractive manufacturing. In aspects, the device may consist of both custom and non-custom elements. For example, a custom foot orthotic may be coupled with an off-the shelf cuff, as well as other variations. The custom fit will ensure user comfort and maximize effectiveness by controlling contact area and resulting force distribution where the device is affixed to the user. For example, the proximal cuff may be designed to be affixed to less sensitive parts of the user's ankle, to avoid scarring or injury, or it may be designed to distribute forces optimally around the ankle while tension is applied on one side to prevent adverse rotation and discomfort. Similarly, the distal portion may be custom-fit based on the intended geometry or support needs of the user under load, as prescribed by a doctor or determined through analysis of user data. For example, a custom foot orthotic may be included with the ankle orthosis based on a scan of the user's foot to improve gait or unload or improve biomechanics of the joint in combination or individually from functional ankle orthosis. Custom design and fabrication may not just determine personalized fit, but also the direction of force across or between the joint. For example, anchor points or channels in the device to guide the tensioning element may be automatically or semi-automatically positioned in the device based on user need, prescription, gait analysis or other data. The overall shape of the device, for example the proximal cuff, may be automatically designed based on scan data, user morphology, radiographic data, prescription data, or user-provided data (e.g. pain within a region of the joint) to automatically adjust forces around and within the joint. Additionally, the adjustment mechanism may be placed optimally using similar data and automated design processes.

The device may be prefabricated and can be further modified by the user or a professional to provide the desired function. Modification may include molding or thermoforming of the rigid components of the device to improve fit, comfort and function. Modifications may further include adjustment of anchor points to create the intended correction or unloading in the individual. The device's modular design can allow a certified fitter or doctor to modify the device's performance based on the specific user need.

The device may be connected to or used in conjunction with additional orthopedic or prosthetic devices using universal or custom connectors. The device may be modular for compatibility with different devices based on the connection required. Other devices which may be connected to the device include a knee orthosis to yield a knee ankle foot orthosis (KAFO), a knee hip orthosis to yield a hip knee ankle foot orthosis, or may connect to prosthetic devices, e.g., prosthetic feet. The device may also be continuously fabricated with such other devices. Any one of these orthoses may contain a similar adjustable tensioning mechanism, which can be used individually or adjust with a single connected adjustment mechanism.

Motors and Sensors

The tensioning or compressive elements may increase or decrease force across, between, or within the proximal and distal portions through adjustment by a motor. The motor may be controlled by an electronic interface connected directly or indirectly to the device. The interface may be an app or software on the user's mobile device, smart watch, or other computer/smart device. In aspects, the device may comprise one or more processors, one or more motors, one or more controllers, one or more sensors, one or more antennas, as well as software to control, instruct, process, command, implement adjustment of the tensioning element(s) of the device.

The motor or elements may control or change force within the tensioning or compression element(s) automatically based on input from sensors located on the device or elsewhere on the user. For example, EMG sensors and/or accelerometers may detect stages of gait to optimize positioning of the foot and resulting gait throughout a range of motion. In another example, sensors may trigger the energy storage units to engage while stepping to support plantarflexion at one stage of gait and support dorsiflexion at another. In other aspects, the tensioning element can be manually adjusted using, for example, a smartphone, smart wearable technology, computer, external processor, or an electronic control mechanism on or attached to the device. These adjustments can be made in real-time or substantially real-time as understood by one of skill in the art; they can be made by the user or a treating practitioner; and/or they can be made while the user is wearing the device. A combination of both automatic and manual adjustment is also contemplated.

Method of Making

The various embodiments of the present disclosure may use traditional manufacturing processes for ankle braces and/or 3D printing/additive manufacturing to produce the components and/or the overall device. These techniques may also be used to fabricate positives through which negative molds are constructed for injection molding.

The fabrication technique allows low-cost, custom devices. It also enables manufacturing of intricate parts containing internal channels and features that could not be feasibly or affordably produced with injection molding, machining, or other traditional methods of manufacturing orthotic devices. 3D printing enables efficient production of lightweight, yet durable materials such as thermoplastics. The result is a highly effective, lightweight, customized, and cost-effective device that can be manufactured at scale.

Dynamic Flexure Joints

In some embodiments, a rigid or semi-rigid upper portion may be connected to the rigid or semi-rigid lower portion of the ankle-foot-orthosis directly by an elastomeric tensioning element. In aspects, the tensioning element acts as a flexure joint between the upper and lower portion. The flexure joint may otherwise be described as a flexible hinge, an elastomeric hinge, a tensioning hinge, or a tensioning joint. As with existing elastomeric flexure joints (for example the Tamarack Flexure Joint®, the elastomeric flexure joint described herein is attached to the distal end of the upper portion and the proximal end of the distal portion in slots or anchors.

The device, in aspects, can be provided as a kit for the assembly with traditional plastic molded AFO's. Alternatively, the device may be incorporated into the design of an AFO digitally and 3D printed. In embodiments described, the support provided by the dynamic flexure joint can be modified using an adjustment mechanism, which allows the user to increase the tensile or compressive force stored within a tensioning or compressive element that is positioned between an upper portion and a lower portion of the AFO.

### BRIEF DESCRIPTION OF THE DRAWINGS

The above and other features, aspects, and advantages of the present invention will be further clarified through the following drawings, detailed description, and claims. The accompanying drawings illustrate certain aspects of some of the embodiments of the present invention and should not be used to limit or define the invention. Together with the written description the drawings serve to explain certain principles of the invention.

FIG. 1 illustrates an anatomy of an ankle joint according to embodiments described herein and designates three axes of rotation: the coronal (ML) axis, the sagittal (AP) axis, and the vertical axis.

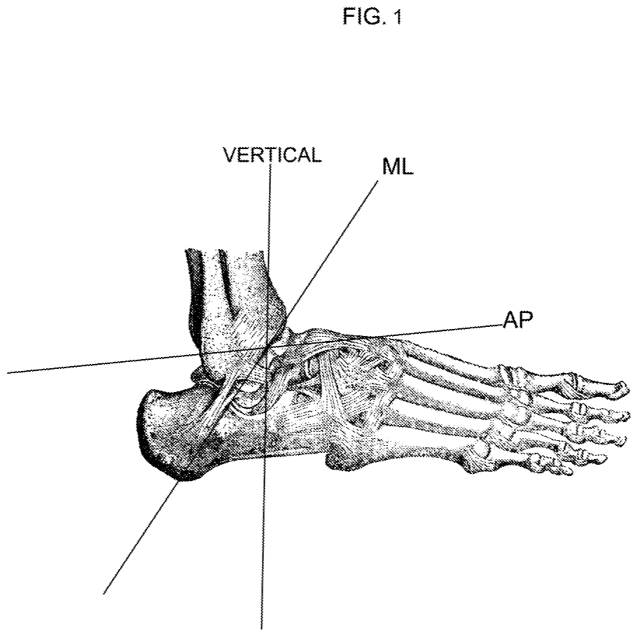

FIG. 2 illustrates a lateral view of a rotation control orthosis comprised of a proximal cuff and a distal portion, connected by an adjustable tensioning apparatus. The tensioning apparatus is comprised of a rotary adjustment mechanism, and cables attached to a tensioning element, which is anchored in the distal portion.

FIGS. 3A and 3B display a lateral view of two variants of a multi-axis rotation control ankle orthosis comprised of a proximal cuff and a distal portion, connected by an adjustable tensioning apparatus. The tensioning apparatus is comprised of an adjustment mechanism, a cable, a tensioning element, and anchors to connect the components. FIG. 3A displays application of the embodiment to prevent inversion while FIG. 3B displays application of the embodiment to limit plantarflexion.

FIGS. 4A-4C illustrates a multi-axis rotation control ankle orthosis comprising a proximal cuff, a sock, and an adjustable tensioning apparatus. The tensioning apparatus is comprised of an adjustment mechanism, cable, and a cable guide, which connects to the sock. FIG. 4B further displays one potential mechanism for adjustable connection of the channel guide to the sock, with a movable hook or loop surface of the cable guide as described in embodiment 3. FIG. 4C displays an application of the embodiment for foot drop or controlling rotation around the ML axis.

FIGS. 5A and 5B displays a lateral view of the ankle orthosis comprised of a proximal cuff and sock and incorporating a tensioning web as described in embodiment 4. FIG. 5A is an application for controlling foot drop, inversion, and or eversion while FIG. 5B is an application for controlling inversion.

FIG. 6A displays a rotation control ankle orthosis comprised of a proximal cuff and an adjustable tensioning apparatus connected by a hinge, and incorporating an adjustable tensioning mechanism. FIG. 6B illustrates an optional slot that may be incorporated into the hinge in FIG. 6B.

FIG. 7 displays an additional distal portion which may be optionally incorporated into the ankle orthoses containing the sock as described in embodiments 3 and 4.

FIG. 8 displays a hook-and-loop opening for the user to easily don and doff the device.

### DETAILED DESCRIPTION OF VARIOUS EMBODIMENTS

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

All references cited in this specification are hereby incorporated by reference in their entireties.

As used herein, the term “proximal” is synonymous with top or upper, as in above the ankle. The term “distal” is synonymous with bottom or lower, as in below the ankle. As used herein, the term “anterior” refers the front of the foot, ankle, or device while “posterior” refers to the back.

Throughout the following detailed description, it should he understood that elements in tension are by example and that the same rotational force may be generated by a compression element on the opposing side of the ankle.

It should also be understood that the adjustable tensioning and compression apparatuses herein are described in ankle orthoses by example only. One skilled in the art would recognize that similar adjustable tensioning and compression apparatuses could be applied to control rotation and forces around other joints including the wrist, shoulder, back, neck, knee, hip, and elbow. For example, the adjustable tensioning apparatus described herein could be used in an assistive knee orthosis.

Throughout the following detailed description, the same reference numbers refer to the same elements in all of the figures.

### EXAMPLES

Preferred Embodiment One: Multi-Axis Rotation Control Ankle Brace with Proximal and Distal Portions and Hinge

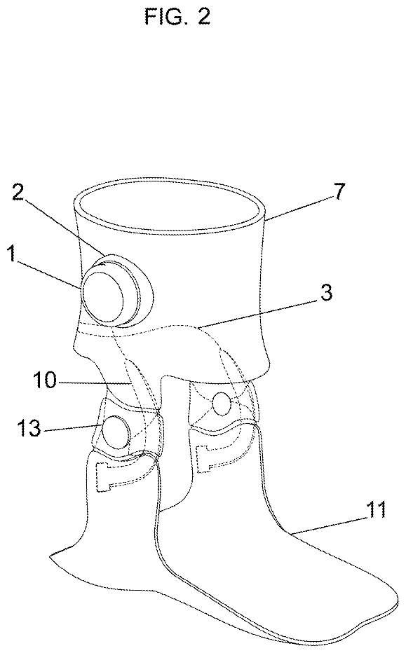

Preferred embodiment one (FIG. 2) is comprised of a proximal cuff (7) and distal portion (11) connected by a hinge (13). The device contains an adjustable tensioning apparatus (3,9,10) that runs across the one or more hinge that is positioned medial and/or lateral to the ankle joint. The tensioning element in this case represents one type of energy storage element, in this case under tension. The tensioning element in this example further comprises a tensioning element (10), a rigid cable (3) and an adjustment mechanism (1). The adjustment mechanism (1), in this case a rotary dial, is embedded in an adjustable mechanism insert (2) within the proximal cuff (7). It may be held in place by the form of a locking mechanism, where the dial's male end is complementary with the female insert in the proximal cuff (7). It may be sewn or glued or otherwise bonded into the proximal cuff. Regardless of the method, the adjustable mechanism insert (2) ensures that the adjustment mechanism (1) is securely in place when subjected to any force subjected by the user intentionally or inadvertently by the user, including but not limited to rotation in either direction, or pulling out. The adjustment mechanism (1) is further connected to the cable (3), for example by a knot. The term cable herein generally refers to any type of cable, wire, braid, string, chain, lace, rope, or article that is substantially flexible yet not extensible and is able to be wound up and transmit tensile forces.

The cable or cables are attached to the one or more tensioning element, which is inserted into the distal portion, and may be anchored in a slot within the distal portion. When tension is applied to the tensioning element, a counter-clockwise torque is generated around the hinge. This torque would generate rotation around the ML axis to support dorsiflexion and resist plantarflexion. One skilled in the art would understand that anchoring the tensioning elements in the opposite orientation around the hinge and generating a clockwise force would have the opposite effect: generating rotations around the ML axis to support plantarflexion and resist dorsiflexion.

The adjustment mechanism (1) allows the user to change the magnitude of force within the tensioning element, and therefore the force around the joint based on their need. In some embodiments, the adjustment mechanism allows tension to be increased in one direction, e.g., by clockwise rotation of a dial. The tension may be released rapidly by disengaging the dial, e.g., by pulling it out, pushing it in, or pressing a button. In other embodiments, the adjustment mechanism may allow gradual increase or decrease of tension. For example, clockwise rotation of a rotary dial would increase tension, while counterclockwise rotation of the dial would reduce tension. The adjustment mechanism may be a rotary dial, a lever, a switch, a ratchet and pawl mechanism, a pulley, or an electronic adjustment mechanism operated by a motor. The adjustment mechanism may he placed in an optimal position for use that is non-obstructive during activity, but accessible, for example at the rear of the proximal cuff.

Preferred Embodiment Two: Multi-Axis Rotation Control Ankle Brace with Proximal and Distal Portions

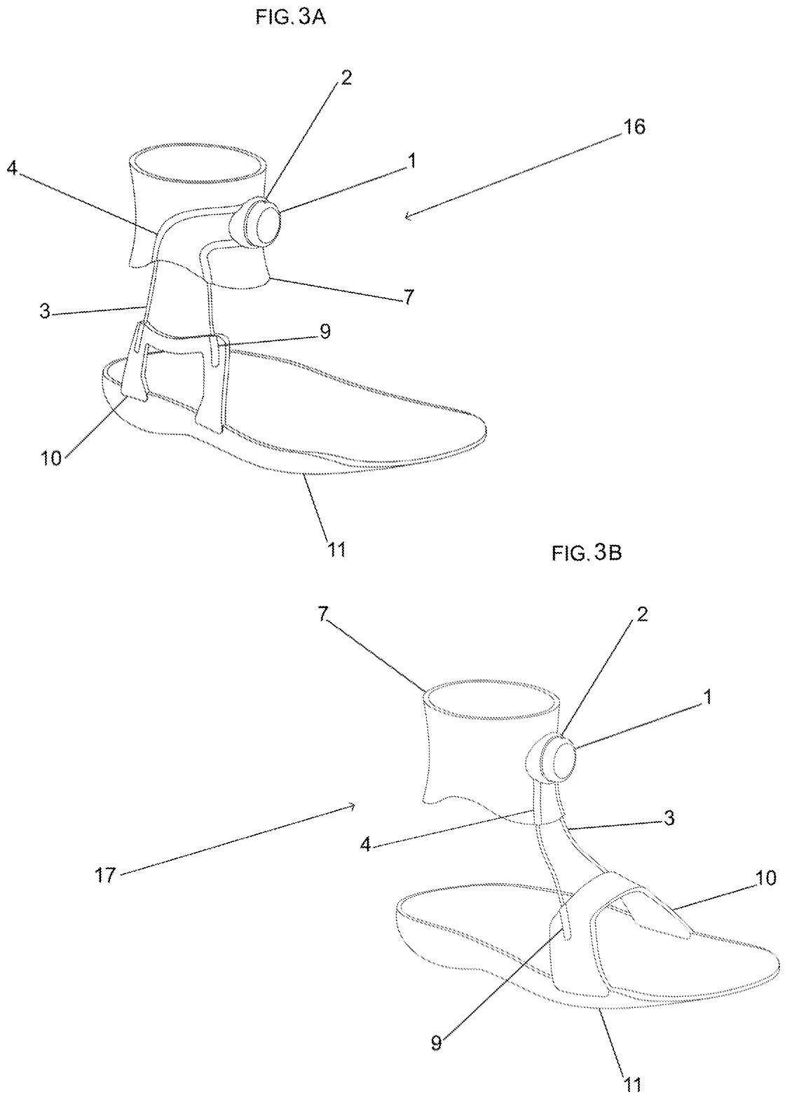

Preferred embodiment two (FIG. 3A) is comprised of a proximal cuff (7) and distal portion (11) connected by a tensioning element (3,9,10) that runs across or near the ankle. Unlike embodiment 1, (FIG. 2), the proximal cuff and distal portion are connected by the tensioning element rather than a hinge. However, the embodiment contains functionally equivalent descriptions of the components of the adjustable tensioning apparatus including the adjustment mechanism (1), the adjustable mechanism insert (2), one or more cable (3), and a tensioning element. The tensioning element in this case represents one type of energy storage element, in this case under tension.

The way the cable or cables are connected to the adjustment mechanism may change how forces are applied on one or more cables, e.g., adding tension to one while decreasing tension on another, or providing equivalent tension to both cables. In the same way, tension may be increased on one side of the cable and decreased on the other side of the cable.

The cables may be connected directly to the distal portion, therefore directly connecting the adjustment mechanism and the distal portion. The cables may also be indirectly attached to either the adjustment mechanism and/or the distal portion. Indirectly attached is herein defined as being connected to described components, e.g. a cable may be indirectly attached to the distal portion by attaching to a tensioning element, wherein the tensioning element is directly attached to the distal portion. In aspects, these may be connected in line with one another. Such an example is shown in this embodiment (FIG. 3A), where the cable is indirectly connected from the adjustment mechanism (1) to the distal portion (11) by a tensioning element (10) and anchors (9).

The anchors, as described herein, generally refer to any type of anchor, hook, hook and loop materials, button, screw, knot, fastener, insert or connecting mechanism that exist between components of the tensioning apparatus, proximal portion, and/or distal portion. The location of the anchors (9) on the tensioning element (10), proximal cuff (7) or distal portion (11) may be changed to control the direction of three around the joint. They may be set during fabrication or may be moved by the user or professional as necessary to change the direction of force.

The tensioning element (10) is connected to distal portion (11). As such, the proximal cuff is connected to the distal portion by the tensioning element, which runs across or near the ankle joint. By example, the tensioning element may be connected to the distal portion with hooks, hook and loop materials, screws, knots, fasteners, inserts or anchors or simply a melding of material with the tensioning element. The tensioning element and the distal unit may also be manufactured as one continuous piece, for example as a 3D printed or molded thermoplastic material.

The adjustment mechanism (1) allows the user to change the magnitude of force within the tensioning element, and therefore the force around the joint based on their need. In some embodiments, the adjustment mechanism allows tension to be increased in one direction, e.g., by clockwise rotation of a dial. The tension may be released rapidly by disengaging the dial, e.g., by pulling it out, pushing it in, or pressing a button. In other embodiments, the adjustment mechanism may allow gradual increase or decrease of tension. For example, clockwise rotation of a rotary dial would increase tension, while counterclockwise rotation of the dial would reduce tension. The adjustment mechanism may be a rotary dial, a lever, a switch, a ratchet and pawl mechanism, a pulley, or an electronic adjustment mechanism operated by a motor. The adjustment mechanism may be placed in an optimal position for use that is non-obstructive during activity, but accessible, for example at the rear of the proximal cuff.

One or more channel (4) in the proximal cuff guides the positioning and orientation of the cables, therefore controlling the direction of force. The channel may be unique to the specific model of brace, or it may be custom to the individual based on the precise direction of support that they require.

By way of example, a proximal fabric (semi-rigid) cuff (7) may anchor the device to the ankle (as shown in FIG. 3A). The proximal cuff may also be connected to the calf or at another position above the ankle joint. The proximal cuff may be rigid or semi rigid to fit optimally to the user's ankle, calf, or any part of the leg. The distal portion (11) will attach or conform to the user's foot or provide additional support and to maximize comfort. By example, the distal portion may comprise a foot orthotic. The distal portion may be comprised of a rigid or semi-rigid custom or off the shelf orthosis, insert, sole, fabric, or shoe. The distal portion may work synergistically with the adjustable tensioning mechanism in order to optimize the joint geometry, modify forces within other lower limb joints such as the knee and hip, and modify gait overall.

The energy storage element, in this example a tensioning element (10) may comprise rigid, semi rigid, elastic or spring elements individually or in combination. Force may be stored within the energy storage element to generate a torque or force around the desired axis or combination of axes of the joint. The modularity of the device enables this axis of rotation to be changed, either in manufacturing, by the clinician or by the user. For example, by positioning the tensioning element on the lateral side of the foot, inversion can be limited or prevented (FIG. 3A). Alternatively, the tensioning element placed anterior to the ankle may prevent foot drop (FIG. 3B). The direction of the net force vector and positioning of forces around the joint to control range of motion may be modified by positioning of the channels or guides within the proximal portion through which the cable or cables run. Additionally, direction of the net force vector and positioning of forces around the joint to control range of motion may be modified by the shape of the tensioning element (10), for example an elastomeric band with one point of connection between the proximal and distal portions vs. an elastomeric web with two points of connection to the distal portion (as shown in, e.g., FIG. 3A) vs. an elastomeric sheet continuously connected along the distal portion would change the distribution and direction of the net force vector.

Overall, the embodiment allows for customization to fit the user's specific need in many aspects. The magnitude of force can be modified by the adjustment mechanism (1) and also by the material properties and geometry of the tensioning element (10). The direction of force and axis of rotation can be modified by the adjustment mechanism, the channel (4) location on the proximal cuff (7), the location and number of anchor points (9) between the cable (3) and the tensioning element (10), the geometry of the tensioning element (10), and/or the location of the connection between the tensioning element (10) and the distal portion (11). Through these mechanisms, rotation can be limited or controlled in one or more of the ML, AP and vertical axes to stabilize, correct, or unload the ankle joint. The device can then therefore be engaged to restore proper foot and ankle orientation or joint geometry.

Preferred Embodiment Three: Multi-Axis Rotation Control Ankle Brace with Sock and Cable Guide

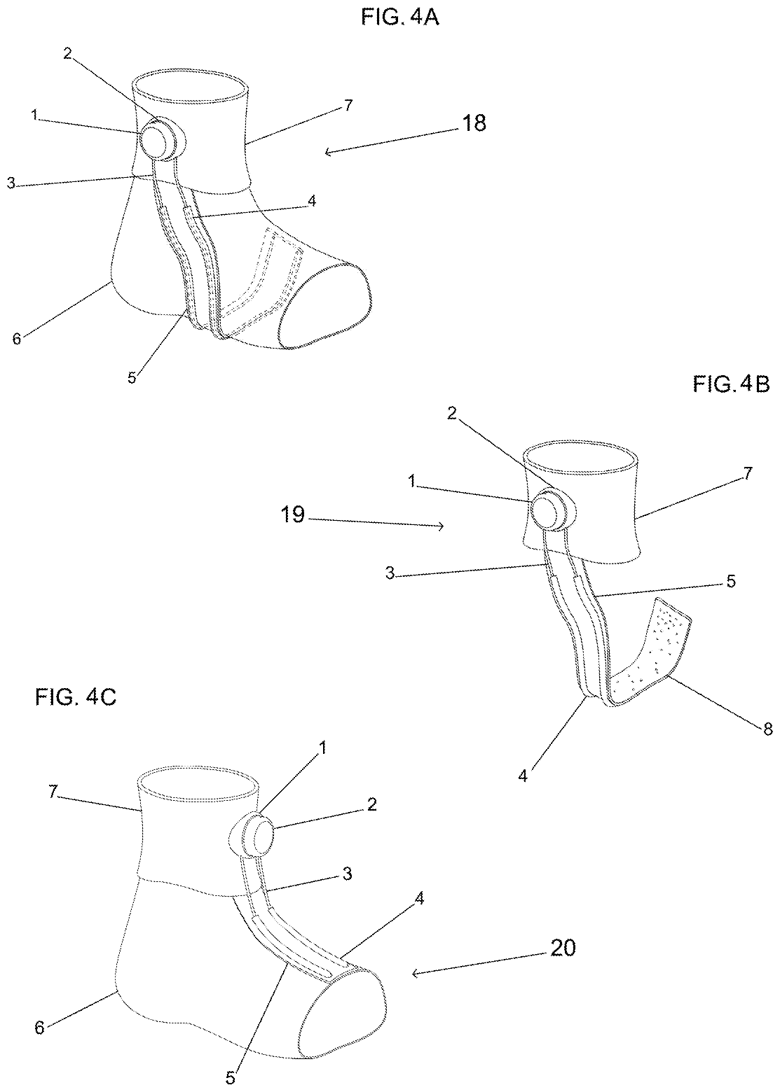

Embodiment three is comprised of a proximal cuff (7) and a sock (6) that incorporates a tensioning element (1,3,4,5). (See, FIG. 4A) The proximal cuff may be connected to the sock e.g. by sewing, hook and loop, hooks, buttons, or other fasteners. The proximal cuff and connected sock may also be one continuously manufactured unit or material. In this embodiment, the tensioning apparatus is comprised of an adjustment mechanism (1), one or more cable (3), one or more channel or pathway, optionally a tensioning element such as an elastic band, and optionally one or more cable guide. The adjustment mechanism (1) and adjustment mechanism insert (2) are the same as described in embodiment 1 (See, FIG. 2). Similarly, the adjustment mechanism may be connected to one or more cables as described in embodiment 1. In the example shown in FIG. 4A, rather than connecting to a tensioning element as described in embodiment 1, the cables are connected to a cable guide (4). The rigid, semi-rigid, or mostly inelastic cable guide connects to the sock (6) in order to direct force around one or more axes. The cable guide may consist of an elastomer or fabric, containing a hook (8) on one side (as shown in FIG. 4B) to adhere to the sock containing loop on its surface, at any position.

Lace or wire may run through the channels (4) in the cable guide (5) and operatively connect to a rotary dial housed on the proximal portion, as described in embodiment one. In this way, the band can be tensioned to generate or limit rotation around the ankle depending on the path of the cable guide. In one embodiment, the band may be positioned on the lateral side of the foot and tensioned to limit or prevent inversion. If the hand is positioned on the lateral side of the foot and in front of the center of the ankle joint (as shown in FIG. 4A), it can generate a torque around 3 axes to 1) limit or prevent inversion, 2) limit or prevent adduction, and/or 3) support dorsiflexion to limit or prevent foot drop; thus, a multi-axis rotation control device. This capability is valuable, as individuals with foot drop suffer to varying degrees with deficiency in dorsiflexion and abduction, not only dorsiflexion. The device, as shown in FIG. 3A would restore the foot of an individual with both of these deficiencies to its normal position and function. If positioned on the top of the foot, the device can prevent, foot drop alone, focusing on dorsiflexion support and correcting rotation around the AP axis.

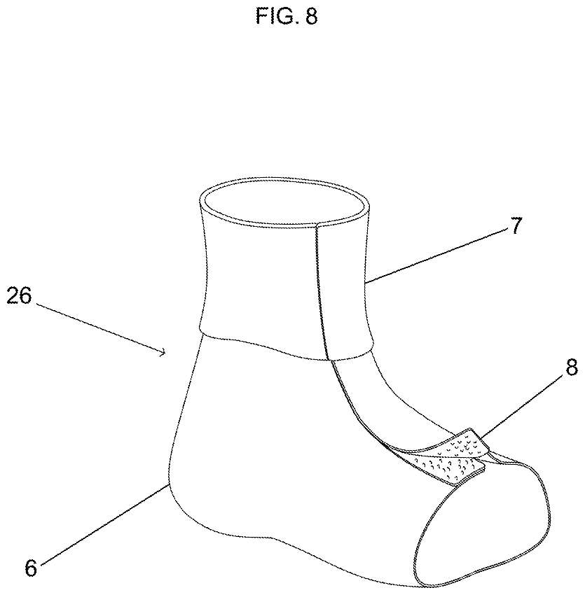

The sock (6) may consist of rigid or semi-rigid materials. By example, it may he produced from a fabric. The fabric may be of universal size for all users and stretch to fit the size of the user's foot, or it may be produced in discrete sizes (e.g. from extra-small to extra-large). The sock may be comprised fully of a loop material, or may be coated with loop material, in order to connect to the hook material (8) on the cable guide (5). The sock may slide onto the joint as a mostly continuous member, or it may unfold and wrap around the joint, with one or more portions coming together to form a sort of sock or sleeve, which may be held together by hook and loop materials, for example as shown in FIG. 8. Other compatible materials may allow temporary or permanent connection between the sock (6) and cable guide (5). In some embodiments, the cable guide may connect at any point and orientation to the sock to precisely determine the direction of force around the 3 axes of the ankle joint. The cable guide may also generate pressure or compression in a target region of the joint based on its position in order to combat swelling, improve joint stability, or relieve pain. In other embodiments, the band can only attach to set positions on the sleeve with compatible material. Specific segments of the sock (6) may contain strips of compatible loop material to which the cable guide (5) can adhere. For example, position 1 may be located as shown in FIG. 4A to limit or prevent inversion for patient A, while position 2 may be located as in FIG. 4C to limit or prevent foot drop, and position 3 on the medial side of the ankle to limit or prevent eversion.

The direction of force may be changed by the position of the cable guide, and/or the orientation of the proximal cuff. For example, the cuff containing the adjustment mechanism may be able to rotate or be attached in any position on the ankle in order to position the tensioning element on the desired side of the ankle. The device may contain multiple tensioning elements, which can be activated dependently or independently.

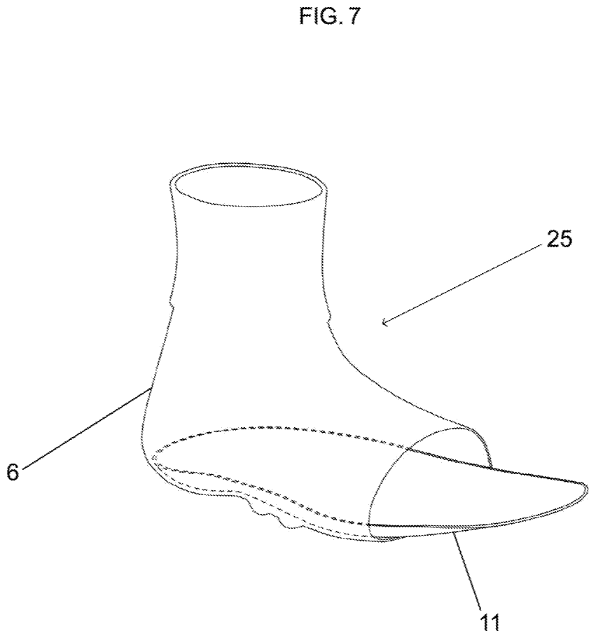

The embodiment containing the cable guide may incorporate custom or off-the-shelf inserts in order to provide further stability or pressure distribution around any region of the foot or ankle. For example, a custom foot orthotic (11) (as shown in FIG. 7), may be incorporated into the sock. By adding tension to the cable guide (FIG. 4A), force will be applied to the foot orthotic insert (11), which will create a distributed force along the user's foot.

Preferred Embodiment Four: Multi-Axis Rotation Control Ankle Brace with Tensioning Web

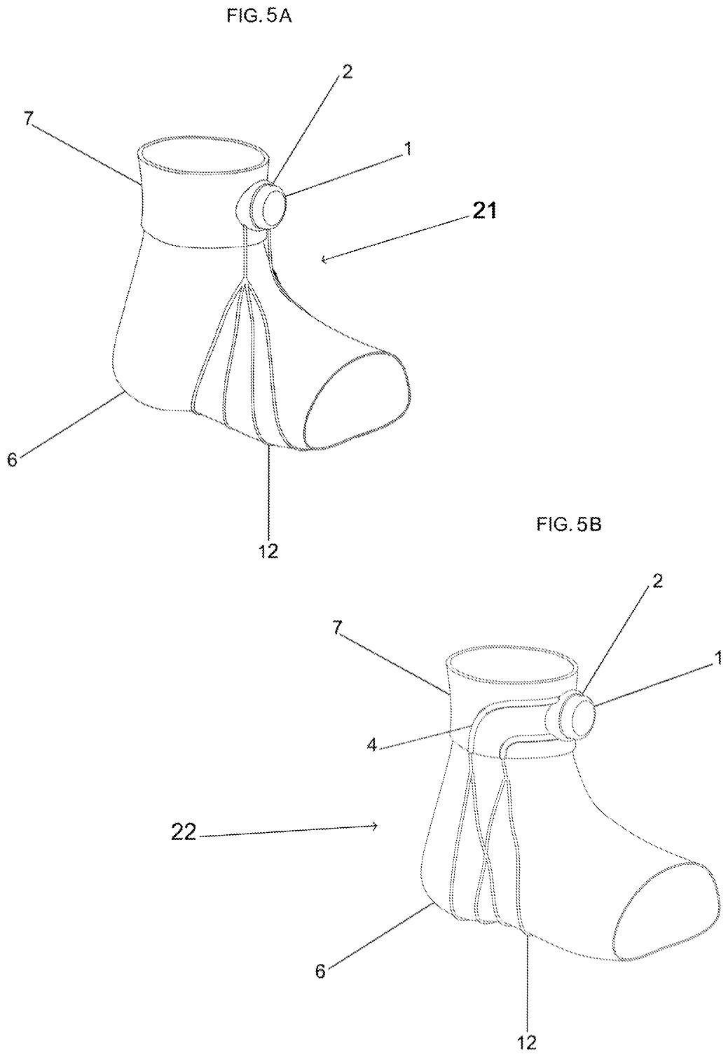

In a fourth embodiment, the brace is comprised of a proximal cuff and sock as shown in FIG. 5A. In this embodiment, the tensioning element is comprised of an adjustment mechanism (1) and a tensioning web (12). The tensioning web may be directly connected to the adjustment mechanism, or it may be connected to the adjustment mechanism indirectly by one or more cable (3), similarly to that described in embodiment 1.

In this embodiment, one or more tensioning web(s) (12) may run along or through the sleeve or sock, for example channels within a fabric. The positioning of the channels guides the direction of force around the joint. As shown in FIG. 5A, the tensioning element(s) may attach directly to an adjustment mechanism, for example a rotary dial, that can increase tension on one or both sides of the foot. In one embodiment (as shown in FIG. 5A), rotation of the dial increases tension equivalently on both sides of the foot. As shown, this would prevent foot drop and also support against both eversion and inversion. Alternatively, the tensioning web can be located on the lateral side of the ankle (as shown in FIG. 5B) to prevent inversion. The dial may increase tension on one side of the foot upon clockwise rotation and decrease tension on the opposite side upon counterclockwise rotation to prevent either inversion or eversion based on the user need. Alternatively, the tension may be disengaged rapidly, e.g. by pulling out or pushing in the dial. The direction of force may be changed based on the positioning of the channels and tensioning elements throughout the sleeve relative to the axes of the ankle. The direction may also be changed by altering the position of channels within the proximal cuff or selecting different channels to feed the tensioning element through, in which the tensioning element is connected to the adjustment mechanism.

Direction and magnitude of force may be customized by the form of the tensioning web itself, including changing the geometry, degree or pattern of branching of the web, the material type, or the material thickness. The tensioning web may be designed to distribute forces in an asymmetric manner or mostly balanced across one or more axes of rotation.

In another embodiment, the sock or sleeve is knitted or stitched or otherwise attached or bonded with materials of different elasticity and or rigidity to allow range of motion in some directions and limit it in others. Regions of the mesh are optionally under static tension which may be optimized for variable directional support. Elastic materials may be weaved together or embedded or attached operationally either inside, outside or within the sock (6). Performance may be adjusted/modified by the geometry, density of mesh, and/or diameter/geometry of the fibers. For example, inversion control may be achieved by having materials of distal elasticity integrated on the lateral side of the sock to restrict rotation in the sagittal plane. Another example is to restrict foot drop and/or promote dorsiflexion plantar flexion by incorporating inelastic mesh in the anterior region of the sleeve while the remaining material remains relatively elastic as to not obstruct normal range of motion in other directions. The device may optionally incorporate a rigid or semi rigid foot orthosis (11), insert, sole or shoe. In some embodiments, the fibers may be directly or indirectly connected to an adjustment mechanism, such as a rotary dial, in order to increase or decrease tension and resulting rigidity of a region of the sock. In others embodiments, the range of motion in a given direction may be controlled statically by the mesh characteristics without required adjustment by the user.

Preferred Embodiment Five: Multi-Axis Rotation Control Ankle Brace with Hinge

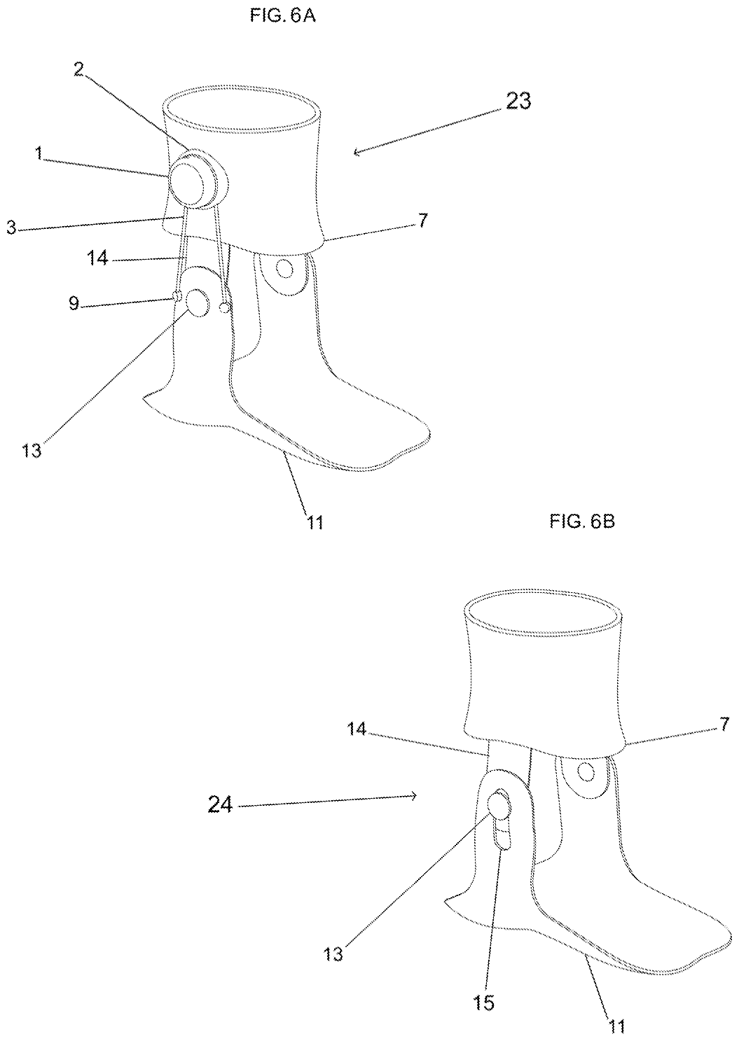

In another embodiment, the brace may optionally contain a hinge on the medial and lateral sides of the ankle joint, which allows rotation around the ML axis, but prevents rotation in the AP and vertical axes (as shown in FIG. 6A). The device contains a proximal cuff (3), one or more vertical member (14), one or more hinge (13), and a distal portion (11). The hinge may optionally contain one or more slot (15) on the medial and/or lateral side of the joint (as shown in FIG. 6B). The example in FIG. 6B would allow for movement around the ML axis and limited inversion, with the range of inversion dependent on the size of the slot. Alternatively, a slot located in the medial side of the joint would prevent inversion, but allow a limited range of eversion. It should he understood that the path of the slot an be modified to allow restricted range of motion around the vertical axis, and/or the AP axis. The hinge may be combined with the adjustable tensioning apparatuses described in preferred embodiments 1-4 as explained herein.

In another embodiment, the hinge may incorporate a pulley to which a rigid or semi-rigid tensioning element is connected (FIG. 6A). In this case, the tensioning element is comprised of an adjustment mechanism (1) and a cable (3) connected to the vertical member (14) around the hinge. In this example, rotating the dial counterclockwise would increase tension in the cable anterior to the hinge center while decreasing tension in the cable posterior to the hinge center. The effect would support dorsiflexion, for example to prevent foot drop. The cables may be anchored around the hinge center at any point on the distal portion (11), or they may be connected directly to the hinge (13) to generate a torque around the hinge center.

Preferred Embodiment 6: Adjustable Elastomeric Hinge

In another embodiment, an upper portion affixed to the user's calf and a lower portion affixed to the user's foot may he connected by an elastomeric hinge, in this case an elastomeric band. In aspects, the hinge may be described as a flexure joint and in this invention specifically, a dynamic flexure joint. The distal end of the elastomeric band is anchored fixedly to the lower portion along the medial side and or the lateral side of the ankle joint. The proximal end of the elastomeric band is fed through a slot or channel in the upper portion. In this embodiment, the elastomeric band includes a stopper (alternatively described as a wedge, further defined as a component of larger cross sectional area or diameter than other regions of the elastomeric band), where when the elastomeric band is inserted into the upper portion, the stopper prevents the elastomeric band from exiting the slot or channel of the upper portion. This stopper allows for a minimal distance of separation, and therefore for maximum degrees of freedom or range of motion of the ankle joint. At the proximal end, the elastomeric band is connected to a cable (defined as a flexible component that does not elongate under tension) to an adjustment mechanism, in this case a dial. In embodiments, the adjustment mechanism winds the cable around a spool and as a result elongates and increases the tension within the elastomeric band. This, in turn, increases the force between the upper portion and the lower portion, drawing them together. In one example of the embodiment, the elastomeric band is aligned on the medial and/or lateral side of the ankle along the joint center. When the device is disengaged (by disengaging the adjustment mechanism), the elastomeric hinge allows full range of motion in all axes, including inversion and eversion. Upon tensioning the device, for example by rotating the dial, tension is stored within the elastomeric hinge to prevent or counteract forces of rotation (for example inversion is prevented if the elastomeric band is located on the lateral side of the device). Just as the stopper allows a minimum amount of tension and maximum distance of separation between the upper and lower portion, the tensioning system may have a maximum set by the channel length, band length, cable length, a clutch on the adjustment mechanism, or other force-limiting mechanism.

In embodiments, the device may comprise a second static elastomeric hinge similar to traditional elastomeric flexure joints, which is positioned at or near the center of the joint. A secondary elastomeric hinge (or elastomeric band) component that is positioned anterior to joint center, is fixedly connected to the lower portion, and is fed through a slot or channel at the upper portion, connected to an adjustment mechanism by a cable. By increasing tension on the dynamic element of the hinge, dorsiflexion support is provided. The amount of tension can be adjusted to meet the user's weight, activity, or stage or rehabilitation without limiting their overall life. Furthermore, the amount of adjustment can accommodate a specific angle of correction for dorsiflexion support or a force required to restore gait without fully limiting plantarflexion. The static hinge controls range of motion of the joint with regards to inversion or eversion (around the AP axis). The elastomeric components and hinges provide an important function to support or augment the joint in a more biomechanically favorable manner, rather than fully stopping range of motion at a discrete point which is generally unnatural.

In other embodiments, one adjustment mechanism may be connected to two elastomeric bands. Both elastomeric bands, as previously described, are fixedly connected to the lower portion and drawn through slots or channels in the upper portion, each optionally comprising a stopper that limits the distance between the upper and lower portions. The first elastomeric band is positioned anterior to the joint center on the medial and/or lateral side while the second elastomeric band is positioned posterior to the join(center on the medial and/or lateral side. In embodiments, the two elastomeric bands are connected and run through a channel in the lower portion or are anchored in a slot on the lower portion. The proximal ends of the elastomeric bands are connected to an adjustment mechanism with cables, in this example a dial. Rotation of the dial in one direction, for example the clockwise direction, will increase tension in the anterior band, therefore providing a clockwise force around the ML axis and positioning the foot into plantarflexion. Alternatively, by rotating the dial in the counterclockwise direction, the tension in the posterior tensioning element is decreased and the tension in the anterior tensioning element is increased, positioning the foot in a dorsiflexed position or providing dorsiflexion support. As with prior embodiments described, the value of the elastomeric component over rigid elements allows for a corrective force without fully limiting the range of motion. The adjustment mechanism allows the user to modify the specific degree of plantarflexion or dorsiflexion support provided by the assistive orthotic device based on their need at the time.

Other embodiments apply the same components to control forces around other axes of the joint. For example, by positioning the tensioning elements around the vertical axis within the upper or lower portion, adduction and abduction can be controlled. In this embodiment, a static flexure join(may connect the upper portion to the lower portion on the medial or lateral side of the ankle near joint center. Positioning the tensioning system to connect the upper and lower portion but in a direction that generates a torque around the vertical axis of the ankle joint may be able to correct abduction or adduction and restore gait. In other embodiments, the tensioning element may be positioned on the posterior end of the device by the heel. When tensioned, this would generate a force to support plantarflexion. In other embodiments, a tensioning system connected to one or more adjustment mechanism may be positioned on both the medial and lateral side of the ankle joint. By tensioning one side of the device with the adjustment mechanism, for example the medial side, eversion can be prevented. By tensioning the lateral side, inversion can be prevented. By tensioning both sides, the ankle can be stabilized overall to prevent eversion or inversion and without limiting range of plantarflexion or dorsiflexion.

In other embodiments, the elastomeric hinge may be in the form of a web or sheet that connects a rigid upper portion to a rigid lower portion and surrounds all or most of the ankle joint. The elastomeric web is fixedly connected to the lower portion with a slot, rivets, screws or other fixtures. The elastomeric web connects to the upper portion with a stopper that provides a maximum distance and minimum tensile force between the upper and lower portion. Regions of the elastomeric web are connected to an adjustment mechanism, for example a dial, with cable. The cable may be laced throughout the proximal end of the elastomeric web.

By rotating the dial, tension in one or more regions of the elastomeric web is increased as the web is drawn up into the upper portion. This in effect will increase the rigidity or resistive force of one or more regions (for example the lateral side) of the hinge element. In doing so, range of motion can be precisely controlled and can be adjusted depending on user weight, activity, morphology, clinical data, or comfort level.

All embodiments may optionally incorporate a rigid hinge element that rotatably connects the upper portion to the lower portion in combination with one or more tensioning elements as described.

Preferred Embodiment 7: Joint Unloading Ankle-Foot-Orthosis Hinge

The described embodiments largely cover systems under tension to control rotational. forces around the joint. In many aspects, adjustable systems under compression may achieve the same outcomes if positioned along opposing sides of the axis of rotation. Additionally, translational forces (linear forces along a plane or along an axis of rotation) may be achieved with tensioning or compression elements. One beneficial application of translational forces across the joint is to decompress, unload, or distract the ankle joint. These terms may be used interchangeably to indicate a force or forces separating the ankle joint or resisting compression within the ankle joint.

One embodiment is capable of providing an unloading (or separating) force between the upper portion and the lower portion of an ankle foot orthosis. The force is adjustable to unload the ankle joint, provide shock absorption, or decompress the joint during gait. The brace is comprised of a rigid or semi-rigid portion affixed above the ankle, for example to the calf of the wearer. The upper portion includes elements or features to ensure an effective fit and prevent migration of the portion in the proximal or distal direction when a force is applied across the ankle joint by the compression mechanism. The brace further comprises a rigid or semi-rigid lower portion affixed below the ankle joint, for example to the foot. The lower portion is designed to prevent migration when subjected to a force in the proximal or distal direction by the compression mechanism The lower portion may partially or fully encapsulate to the top of the foot or the top of the heel. Elements to ensure effective fit and prevent migration of the upper and lower portions include custom fit through molding or custom-3D printing, strap systems, load distributors, silicone padding, or mechanisms for attachment to the wearer's clothing, including footwear.

The upper portion and lower portion are connected directly or indirectly by a compression element which may be one or more elastic components, one or more springs, pneumatic elements, hydraulic elements, or combinations thereof. The compression element is capable of supporting forces under compression of up to 2.9 times body weight or roughly up to 6500 lbs without failure. The compression element is coupled with an adjustment mechanism and a sliding member. The adjustment mechanism, in this example, is a dial. In other embodiments, the adjustment mechanism may be a dial, a lever, a button, a ratchet and pawl system or other user-operable mechanisms of increasing or decreasing forces within the compression element. In this embodiment, the adjustment mechanism is connected to a slide member. The slide member is housed within a slot of the upper portion, allowing the slide member to move in a proximal and distal direction within the upper portion. Upon adjustment, for example rotation of a dial, the slide member is pushed in a distal direction. The slide member, in turn, applies a force to the compression element. In aspects, the compression element may be connected to the slide member on the proximal end and connected to the lower portion on the distal end. Movement of the slide member in the distal direction further compresses the compression element when under load, and causes the upper portion and the lower portion to separate. By further increasing the compressive force stored within the compression element, the overall unloading force applied to the joint is increased. In other embodiments, the device may further comprise a coupling mechanism, which translates a rotational force generated by the adjustment mechanism to a linear or translational three that drives the slide member. The coupling mechanism may comprise a linear gear system, a spring mechanism, a pulley mechanism, one or more worm gear, planetary gear systems, or other mechanisms that translate rotational forces to linear forces. For example, the adjustment mechanism may be a dial coupled to a spool and optionally comprising a planetary gear system. By rotating the dial, the spool winds a cable which is routed around a wheel or dowel at the distal end of the upper portion and is tied to the proximal end of the sliding member. By rotating the dial, the spool winds the cable and translates the sliding member through a slot in the upper portion in the distal direction. In another example, the adjustment mechanism is a dial, which is coupled to a pinion gear. Upon rotation, the pinion gear drives a linear gear rack, which is further coupled to the sliding component, to translate the sliding component in the distal direction through a slot within the upper portion.

In other embodiments, the unloading ankle-foot orthosis may comprise additional shock absorbing or energy storing elements connected to or fabricated with the lower portion including springs, carbon fiber footplates, pneumatic elements, gels, or elastomers. In some embodiments, the lower portion is the user's footwear.

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection. In another embodiment, the user interface may be managed and controlled through an App or program on a phone, tablet, or other portable electronic device.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all the references cited in this disclosure are each individually incorporated by reference herein in their entireties and as such are intended to provide an efficient way of supplementing the enabling disclosure of this invention as well as provide background detailing the level of ordinary skill in the art.

### Methods of Use

The device in embodiments 1-5 are designed to maximize ease of use and comfort for the user. The device is comprised of lightweight, durable materials including fabrics, plastics, elastomers, and 3D printed thermoplastics. In aspects, the user can easily and rapidly (within 10 seconds) don and doff the device. In some embodiments, the proximal cuff and sock may be flexible so that the user can easily stretch the material to slide their foot and ankle into the device. In other embodiments, the device may contain an opening (as shown in FIG. 8), for example made of hook (27) and loop, so that the user can quickly open and close the device to don and doff. The device can be manufactured to have a low profile, in order to fit comfortable and conveniently within the user's footwear. Alternatively, the device may connect to the user's footwear internally or externally. In other embodiments, the device itself may substitute for the user's footwear and function as a shoe with an integrated tensioning mechanism.

In the various embodiments of the present disclosure, the magnitude and direction of force applied (or resistance or tension generated in the brace) can readily be tailored to the user based on their size, weight, joint geometry, injury, and desired activity. Braces as described herein can be light-weight, robust, low-profile, and well-fitting to users. Unlike braces in the prior art, those disclosed herein can be narrow and lightweight to be worn within the shoe, which is required for high-performance athletics but is not enabled by many existing braces on the market.

The various embodiments of the ankle brace of the present disclosure can be used, by way of non-limiting examples: prophylactically to prevent injury; to reduce joint pain (e.g. during physical exercise or athletic competition); to rehabilitate existing injuries; post-operatively (high tension braces to immobilize the joint to a comfortable level within one or more desired range of motion); to assist or augment movement within a desired direction (e.g. in a direction limited by muscle, tendon or ligament deficiency or damage), and to provide stability and alignment to the joint.

The device described in embodiments (16, 17, 18, 20, 21, 22, 23) can correct the position of the foot relative to the user's ankle to restore proper gait, improve user's stability and prevent falls. For example, the user can address foot drop through the method of 1) sliding their foot through the proximal cuff (7), in some embodiments, sliding their foot through the sock (6), 2) optionally adjusting the position of the tensioning mechanism as described in the embodiments (16, 17, 18, 20, 21, 22, 23), which will determine the effective axis of rotation for correction, and 3) adjusting the tension on the device to apply a force around the effective axis of rotation, which will determine the degrees of corrective rotation around that axis. For example, a user with foot drop would don the device, and rotate the dial, by example, one rotation to increase dorsiflexion by 3° around the ML axis, therefore restoring the angle of the foot to normal (e.g. 90° offset from the vertical axis).

In other uses, the user may apply the device in embodiments (16, 17, 18, 20, 21, 22, 23) to limit rotation of the joint within one or more directions. For example, an individual with chronic ankle instability (CAI) leading to frequent inversion, would 1) don the device, for example shown in FIG. 3A, 2) optionally adjust the position of the device using the anchor (9) positioning, and 3) use the adjustment mechanism (1) to apply force on the lateral side of the ankle, therefore limiting rotation about the AP axis and stabilizing the ankle.

In other uses, the user may apply the device in embodiments (16, 17, 18, 20, 21, 22, 23) to augment movement of the joint in one or more directions. For example, an athlete intending to increase plantarflexion force of would use a device similar containing an elastomeric tensioning element on the posterior side of the ankle (aligned with the Achilles tendon). The user would 1) don the device, for example the device shown in FIG. 3A, 2) optionally adjust the position of the device, and 3) increase force in the tensioning element using the adjustment mechanism to resist dorsiflexion but augment plantarflexion. Such an application could increase the force of plantarflexion, for example to increase the strength and speed of a runner.

In other uses, the user may apply the device in embodiments (16, 17, 18, 20, 21, 22, 23) to support recovery or improve range of motion of the muscle, ligament, tendon, cartilage, and/or bone of the foot/ankle post-operation or post-injury. Additionally, the user may use the device to maintain joint function during the recovery period while protecting the joint from further damage. For example, for a user with lateral tendon tear, the user would 1) don the device, for example shown in FIG. 4A, 2) optionally adjust the position of the device, and 3) increase force in the tensioning element using the adjustment mechanism to prevent inversion while allowing range of motion in other axes, therefore protecting the lateral ligaments from further strain.

The embodiments described (16, 17, 18, 20, 21, 22, 23) are capable of unloading a region of the ankle joint based on the direction and magnitude of force generated by the tensioning element. This can be achieved in one aspect by generating a counterforce on one side of the ankle to reduce contact force on the opposite side. For example, by engaging an elastomeric tensioning element on the medial side of the user's ankle, the lateral side of the ankle will be separated. This will separate the lateral side of the ankle, and act as a spring to reduce the force within that region of the joint upon impact with the ground at each step. The mechanism can also reduce forces in an osteoarthritic or damaged region of the joint by altering the user's gait. For example, by engaging the tensioning element to support dorsiflexion, generating a torque around the ML axis of the ankle, the posterior region of the ankle will contact the ground force and experience the initial impact. In this way, the anterior region of the ankle can be shielded from more significant forces that the user would normally experience without use of the device. In the case of an individual with ankle OA in the anterior region of their joint, this would significantly reduce their pain during activity.

The device is designed such that it may remain in the user's footwear after removal, or it may remain attached to the user and removed from the footwear for use without the footwear. In other embodiments, the proximal cuff (7) and/or sock (6) may be opened and closed around the user's foot for attachment (see FIG. 8).

These embodiments may also be equipped with at least one sensor that collects useful information on gait, biomechanics, and health of the joint. This information may then be used to tailor or tune the device more precisely to the needs of the user, or inform a medical professional of information helpful in making decision on a rehabilitation or treatment regimen, including decisions related to surgical procedures and joint implants. The device can also augment the function and performance of the joint post-operatively and compliment the function of an implanted device or component, actively or passively.

It should be understood that the described embodiments are by example only. The same embodiments may incorporate springs or other energy storage elements to generate force across the joint in a similar fashion to the tensioning elements described. Additionally, adjustable compression mechanisms may substitute for the adjustable tensioning mechanisms in order to perform similar functions as described in the multi-axis rotation control brace.

1. An ankle orthosis wearable relative to an ankle and a foot of a wearer, the ankle orthosis comprising:
a proximal portion affixable above the ankle of the wearer;
a distal portion extendable below the ankle of the wearer; and
a flexible or semi-flexible joint connecting the upper portion and the lower portion, further comprising:
a tensioning element; and
an adjustment mechanism;
wherein the tensioning element generates a rotational force between the upper and lower portion around one or more that one axis of rotation of the joint and wherein the wearer can adjust the magnitude or direction of rotational force while the orthotic device is being worn.

2. The ankle foot orthosis of claim 1 further comprising a hinge, wherein the hinge rotatably connects the upper portion and the lower portion.

3. The ankle foot orthosis of claim 1 wherein the tensioning element is an elastomer or spring and wherein the adjustment mechanism is a dial, which are connected by a cable.

4. The ankle foot orthosis of claim 3, wherein the one or more tensioning element connects the upper portion to the lower portion of the ankle-foot orthosis on the medial side, the lateral side, or both, wherein the one or more tensioning element is located anterior to the ankle joint center, and wherein when the device is tensioned, a counterclockwise rotational force is generated around the ML axis.

5. The ankle foot orthosis of claim 3, wherein the one or more energy storage element connects the upper portion to the lower portion of the ankle-foot orthosis on the medial side, the lateral side, or both, wherein the one or more energy storage element is located posterior to the ankle joint center, and wherein when the device is tensioned, a counterclockwise rotational force is generated around the ML axis.

6. The ankle foot orthosis of claim 1 wherein the adjustment mechanism is a dial, a lever, a ratchet-pawl system, a pulley system, a linear gear system, or combinations thereof.

7. A flexible or semi-flexible joint assembly for an orthotic device comprising
an energy storage element connected directly or indirectly to an adjustment mechanism, wherein a force provides a rotational force around one or more axis of rotation of the joint and wherein the wearer can adjust a magnitude of rotational force while the orthotic device is being worn.

8. The flexible or semi-flexible joint of claim 7 wherein the energy storage element is a tension or compression element.

9. The flexible or semi-flexible joint of claim 8 wherein the tensioning or compression element is comprised of one or more elastomer, spring, hydraulic spring, pneumatic spring, magnetic spring, or combinations thereof.

10. The flexible or semi-flexible joint of claim 7 wherein the adjustment mechanism is a dial, a lever, a ratchet-pawl system, a pulley system or combinations thereof.

11. An ankle foot orthosis comprising
an upper portion affixed to a body part above the ankle joint;
a lower portion affixed to the foot;
a joint connecting the upper portion and the lower portion that optionally articulates;
a sliding component housed within the upper portion that contacts the joint on the lower portion; and
an adjustment mechanism;
wherein the adjustment mechanism is capable of translating the sliding component in a proximal or distal direction, and wherein the resulting force generated by the sliding member provides a translational force between the upper portion and the lower portion, a and wherein the wearer can adjust the magnitude of translational force while the orthotic device is being worn.

12. The ankle foot orthosis according to claim 11, wherein the adjustment mechanism is a dial, and wherein rotation of the dial causes the sliding component to move in a proximal or distal direction.

13. The ankle foot orthosis according to claim 11, further comprising a coupling mechanism, wherein the coupling mechanism converts a rotational force generated by the adjustment mechanism to a linear force, which causes translation of the sliding member in the proximal or distal direction.

14. The ankle foot orthosis according to claim 13, wherein the coupling mechanism comprises a linear gear system, a pulley system, a worm gear, or combinations thereof.

15. The ankle foot orthosis according to claim 11, wherein the translational force is applied along one or more axes of the ankle-joint, and wherein the translational force is capable of reducing forces in between bones of the ankle.

16. The ankle foot orthosis according to claim 11 wherein the adjustment mechanism comprises tensioning dial including an elastomer or spring, and wherein the translational force is increased or decreased by a sliding member, which is operated via the adjustment mechanism to increase or decrease the force between the upper and lower portion and thereby increase or decrease the compressive force within the one or more elastomer or spring while the orthosis is load bearing.

17. The ankle foot orthosis according to claim 11 further comprising a compression element, wherein the compression element is connected to the sliding member on the proximal end, is connected to the lower portion on the distal end, and wherein the compression element is capable of storing energy in compression when subjected to a force.

18. An ankle-foot orthosis comprising
an upper portion that primarily makes contact with the lower leg; and
a lower portion that primarily makes contact with the foot;
wherein the upper portion and the lower portion may be continuous;
wherein the orthosis approximately conforms to the shape of the limb of a user;
wherein the thickness, height, width, and curvature of at least one portion of the orthosis are modified to have mechanical properties that produce a return of force into the limb during ambulation that are within a desired range of force values;
wherein the forces may change on or more axes during ambulation.

19. The ankle-foot orthosis in claim 18 where the mechanical properties include, but are not limited to shear strength, tensile strength, stiffness, and flexibility.

20. The ankle-foot orthosis in claim 18 where part or all of the orthosis is 3D printed and partially conforms to the limb, and is optionally based on a 3D scan of a limb or a model of a limb.

21. The ankle-foot orthosis in claim 18 where the orthosis has a carbon fiber component supporting the orthosis, and optionally connecting the upper portion and the lower portion, wherein the position of the upper portion and lower portion are adjustable relative to one another.

22. The ankle-foot orthosis in claim 18 where the upper portion and the lower portion of the orthosis are capable of generating translation along one or more axes to relieve pressure in the foot and or ankle.
